(19) United States
(12) Patent Application Publication
(10) Pub. No.: US 2019/0119191 A1
(43) Pub. Date: Apr. 25, 2019

(54) PROCESS FOR CONVERTING PLASTIC INTO WAXES BY CATALYTIC CRACKING AND A MIXTURE OF HYDROCARBONS OBTAINED THEREBY

(71) Applicant: SOLVAY SA, Brussels (BE)

(72) Inventors: Stéphane STREIFF, Shanghai (CN); Dominique BALTHASART, Brussels (BE); Marco PICCININI, Brussels (BE); Michel GARRAIT, Charly (FR); Avelino CORMA, Valencia (ES); Miriam CERRO-ALARCÓN, Valencia (ES); Jesús MENGUAL, Carcaixent (ES)

(21) Appl. No.: 16/089,468

(22) PCT Filed: Mar. 31, 2017
(86) PCT No.: PCT/EP2017/057653

§ 371 Date: Sep. 28, 2018

Related U.S. Application Data
(60) Provisional application No. 62/315,928, filed on Mar. 31, 2016.

(30) Foreign Application Priority Data
Dec. 7, 2016 (EP) 16306636.8

(51) Int. Cl. C07C 51/16; C10G 57/00; C08L 91/08

(52) U.S. Cl. CPC C07C 51/16; C10G 57/00; C08L 91/08; C10G 2400/22; C10G 2300/201; C10G 2300/4012; C10G 2300/1003; C10G 2300/4025; C10G 2300/4018; C10G 2300/4006

(57) ABSTRACT

The present invention relates to a process for converting plastic into waxes by catalytic cracking. The process comprises the steps of introducing the plastic and a catalyst within a reactor; allowing at least a portion of the plastic to be converted to waxes, the waxes being part of a pyrolysis gas formed within the reactor; and removing a product stream containing said waxes from the reactor. The invention also relates to a mixture of hydrocarbons obtainable by that process.

This application claims priority to U.S. provisional application No. 62/315,928 filed on Mar. 31, 2016 and to European application No. 16306636.8 filed on Dec. 7, 2016, the whole content of each of these applications being incorporated herein by reference for all purposes.

### TECHNICAL FIELD

The present invention relates to a process for converting plastic into waxes by catalytic cracking. The process comprises the steps of introducing the plastic and a catalyst within a reactor; allowing at least a portion of the plastic to be converted to waxes, the waxes being part of a pyrolysis gas formed within the reactor; and removing a product stream containing said waxes from the reactor.

The invention also relates to a mixture of hydrocarbons obtainable by that process.

### PRIOR ART

In view of the increasing importance of polymers as substitutes for conventional materials of construction, such as glass, metal, paper and wood, the perceived need to safe non-renewable resources such as petroleum and dwindling amounts of landfilled capacity available for the disposal of waste products, considerable attention has been devoted in recent years to the problem of recovering, reclaiming, recycling or in some way reusing waste plastic.

It has been proposed to pyrolize or catalytically crack the waste plastic so as to convert high molecular weight polymers into volatile compounds having much lower molecular weight. The volatile compounds, depending on the process employed, can be either relatively high-boiling liquid hydrocarbons useful as fuel oils or fuel oil supplements or light- to medium-boiling carbon atoms useful as gasoline-type fuels or as other chemicals. Furthermore, the volatile compounds can be or at least can include waxes.

Catalytic cracking of a mixed waste plastic is a process well-known to the person skilled in the art. For example, U.S. Pat. No. 5,216,149 discloses a method for controlling the pyrolysis of complex waste stream of plastics to convert such stream into useful high-value monomers or other chemicals, by identifying catalyst and temperature conditions that permit decomposition of a given polymer.

Research has been conducted in an effort to optimize process parameters with respect to an increased yield of desired cracking products. For example, US 2015/0247096 A1 describes a method for converting a waste plastic to wax by adding hydrogen to the reaction chamber and heating the waste plastic and hydrogen sufficiently to thermally depolymerize the waste plastic to form a wax product, comprising paraffin and olefin compounds. Cracking is conducted at a temperature of about 300° C. to about 500° C. for a duration of about 1 minute to about 45 minutes, sufficient to cause thermal degradation of substantially all of the melted plastic feed stock.

U.S. Pat. Nos. 6,150,577 and 6,143,940 disclose a method for making a heavy wax composition from waste plastics in a pyrolysis zone at sub-atmospheric pressure forming a pyrolysis zone effluent including 1-olefins and n-paraffins. Pyrolysis is conducted at a temperature of from 500 to 700° C.

M. Arabiourrutia et al. describe in Journal of Analytical and Applied Pyrolysis 94 (2012) 230-237 the characterization of waxes obtained by the pyrolysis of polyolefin plastics. The authors investigated the influence of the cracking temperature on the yields of waxes and volatiles obtained from different polyolefins. It was found that increasing the cracking temperature from 450° C. to 600° C. results for example for LDPE (low-density polyethylene) in a decrease in the obtained waxes from 80 wt. % to 51 wt. %. At the same time the amount of obtained volatiles increases from 20 wt. % to 49 wt. %. These findings support the general understanding that increasing the temperature favors the cracking rate towards the formation of shorter chain products.

M. del Remedio Hernandez et al. describe in Journal of Analytical and Applied Pyrolysis 78 (2007) 272-281 the catalytic flash pyrolysis of HDPE in a fluidized bed reactor at 500° C.-800° C. According to the authors, the presence of low amounts of zeolite led to a significant reduction of the saturated and unsaturated condensable hydrocarbons, while it favored the formation of aromatics and branched paraffins.

There is, however, still a need for further improving the cracking of plastic in particular with respect to the yield of specific products, such as waxes and the composition of such products. In certain applications it is, for example, desirable to obtain a high yield of waxes from plastic. Furthermore, it can be desirable to obtain waxes having an increased average carbon chain length, branched carbon chains, low aromatics and/or an elevated drop point.

### BRIEF DESCRIPTION OF THE INVENTION

The present inventors now found that contrary to the above expectation that with increasing cracking temperature the yield of waxes is decreasing, the yield of waxes is surprisingly increased even at high cracking temperatures and even in the presence of a cracking catalyst if the pyrolysis gas which is formed during the cracking of the plastic and which contains the volatile cracking products has only a short residence time at a temperature above 370° C. Furthermore, the present inventors found that under the specific process conditions a novel mixture of hydrocarbons comprising predominantly waxes is obtained wherein the hydrocarbons have a high number of carbon atoms, are predominantly branched hydrocarbons and have a unique content of alpha-olefins.

### DETAILED DESCRIPTION OF THE INVENTION

The present invention therefore relates to a process for converting plastic into waxes by catalytic cracking, the process comprising:

introducing the plastic and a catalyst within a reactor;

allowing at least a portion of the plastic to be converted to waxes, the waxes being part of the pyrolysis gas formed within the reactor; and

removing a product stream containing said waxes from the reactor;

characterized in that the pyrolysis gas has a residence time at a temperature above 370° C. of less than 60 seconds and the weight ratio of catalyst to plastic introduced within the reactor is at least 0.1:30.

The present invention furthermore relates to a mixture of hydrocarbons, characterized in that the hydrocarbons exhibit a cumulative distribution of their number of carbon atoms such that 20≤d20 and 50≥d50;

≥50 mol % of the hydrocarbons are branched hydrocarbons;

and ≤20 mol % of the unsaturated hydrocarbons are alpha-olefins.

Furthermore, the present invention relates to a process for producing said mixture wherein in the above described process for converting plastic into waxes said mixture is obtained by removing the product stream containing the waxes from the reactor.

In the catalytic cracking of plastic several fractions of chemical compounds are obtained. Usually, there is a gas fraction containing light-weight chemical compounds with less than 5 carbon atoms. The gasoline fraction contains compounds having a low boiling point of for example below 150° C. This fractions includes compounds having 5 to 9 carbon atoms. The kerosene and diesel fraction has a higher boiling point of for example 150° C. to 359° C. This fraction generally contains compounds having 10 to 21 carbon atoms. The even higher-boiling fractions are generally designated as heavy cycle oil (or HCO) and waxes. In all these fractions, the compounds are hydrocarbons which optionally comprise heteroatoms, such as N, O, etc. “Waxes” in the sense of the present invention therefore designate hydrocarbons which optionally contain heteroatoms. In most cases, they are solid at room temperature (23° C.) and have a softening point of generally above 26° C. A definition of the obtained fractions is provided in the experimental section below.

A plastic is mostly constituted of a particular polymer and the plastic is generally named by this particular polymer. Preferably, a plastic contains more than 25% by weight of its total weight of the particular polymer, preferably more than 40% by weight and more preferably more than 50% by weight. Other components in plastic are for example additives, such as fillers, re-enforcers, processing aids, plasticizers, pigments, light stabilizers, lubricants, impact modifiers, antistatic agents, inks, antioxidants, etc. Generally, a plastic comprises more than one additive.

Plastics used in the process of the present invention include polyolefins and polystyrene, such as high-density polyethylene (HDPE), low-density polyethylene (LDPE), ethylene-propylene-diene monomer (EPDM), polypropylene (PP), and polystyrene (PS). Mixed plastics mostly constituted of polyolefin and polystyrene are preferred.

Other plastics, such as polyvinyl chloride, polyvinylidene chloride, polyethylene terephthalate, polyurethane (PU), acrylonitrile-butadiene-styrene (ABS), nylon and fluorinated polymers are less desirable. If present in the plastic, they are preferably present in a minor amount of less than 50% by weight, preferably less than 30% by weight, more preferably less than 20% by weight, even more preferably less than 10% by weight of the total weight of the dry weight plastic.

Preferably, the plastic comprises one or more thermoplastic polymers and is essentially free of thermosetting polymers. Essentially free in this regard is intended to denote a content of thermosetting polymers of less than 15, preferably less than 10 and even more preferably less than 5% by weight of the plastic starting material.

Usually, waste plastic contains other non-desired components, namely foreign materials such as paper, glass, stone, metal, etc.

The plastic used in the process of the present invention can be selected among:

single waste plastic, single virgin plastic on spec or off spec, mixed waste plastic, rubber waste, organic waste, biomass or a mixture thereof. Single plastic waste, single virgin plastic off spec, mixed waste plastic, rubber waste or a mixture thereof are preferred. Single virgin plastic off-spec, mixed waste plastic or a mixture thereof particularly preferred. Mixed plastic waste gives usually good results.

Limited quantity of unpyrolysable component such as water, glass, stone, metal and the like as contaminant of inlet raw material are acceptable. A “low content” preferably means a minor amount of less than 50% by weight, preferably less than 20% by weight, more preferably less than 10% by weight of the total weight of the dry plastic. Preferably, the individual content of any less desirable plastic is less than 5% by weight, more preferably less than 2% by weight based on the total weight of the dry plastic.

Preliminary to the pyrolysis, the raw material can be pretreated by a physico-chemical process including one or more operations as size reduction, grinding, shredding, screening, chipping, metal removal, foreign material removal, dust removal, drying, degasing, melting, solidifying and agglomerating.

The pretreatment can be conducted at a temperature lower or equal to 350° C., preferably lower or equal to 330° C. Gaseous degradation products occurring during the pretreatment are advantageously removed. Examples of gaseous degradation products are hydrochloric acid, hydrobromic acid, hydrofluorhydric acid, CO, CO₂, small carbon containing molecules with no more than 4 C-atoms such as methane, ethane, ethylene, acetylene, propane, butane, propylene, butene, methanol, formic acid, formaldehyde, acetic acid, acetaldehyde, ethanol, acetone and the like.

Some of these degradation products might react with other materials present in the plastic and produce undesired reaction products. Examples of such materials are fillers, for instance alkaline fillers such as PCC (precipitated calcium carbonate) or chalk, lime, soda lime, sodium carbonate, sodium bicarbonate, alumina, titanium oxide, magnesium oxide, calcium oxide, and the like.

At the outlet of the pretreatment, the raw material can be solid or melted, preferably melted. Optionally, an acid capturing component can be added for the pretreatment. Examples of acid capturing components are fillers, for instance alkaline fillers such as PCC (precipitated calcium carbonate), alumina, titanium oxide, magnesium oxide, calcium oxide, and the like.

The present inventors surprisingly found that waxes can be obtained at high yield from the catalytic cracking of plastics even at high de-polymerization temperature if the pyrolysis reactor and the operation conditions ensure a short residence time of less than 60 second of the pyrolysis gas at a high temperature of above 370° C. This finding is particularly surprising because a high cracking temperature should favor cracking rate towards the formation of shorter-chain products in particular in the presence of a catalyst. In other words, lower content of the wax mixture should be found at higher temperatures.

Additionally, the present inventors found that at short residence time of the pyrolysis gas at high temperature not only more of the wax mixture is produced but also the average carbon chain length of the wax is increased. This allows carrying out the cracking process at rather high temperatures which ensures high conversions of the raw material and a low residue of for example less than 50 g/kg plastic raw material but nevertheless allows the production of an increased amount of waxes having even an increased average carbon chain length.

Furthermore, the obtained waxes turned out to be high-value waxes comprising branched hydrocarbons, low aromatic contents and optionally an elevated drop point. The invention therefore also relates to waxes obtainable by the process described herein.

In the cracking reactor the plastic is generally present in a molten state at cracking temperature. The cracking reaction results in a de-polymerization of the plastic yielding lower molecular weight products. At the temperature in the cracking reactor these lower molecular weight products evaporate forming a pyrolysis gas within the reactor. This gas comprises the volatile pyrolysis fractions, such as light-weight hydrocarbons, diesel, kerosene and waxes. The invention is based on the finding that the pyrolysis gas should be quickly removed from the hot cracking reaction zone and it was found that high yields of waxes are obtained if the pyrolysis gas has a residence time at a temperature above 370° C. of less than 60 seconds, preferably less than 50 seconds, more preferably less than 40 seconds, even more preferably less than 30 seconds, furthermore preferably less than 25 seconds and most preferably less than 20 seconds, such as less than 15 or even less than 10 seconds.

On the other hand, cracking reactions still occur in the pyrolysis gas as long as the temperature is high enough. Therefore, if the residence time of the pyrolysis gas at a temperature above 370° C. is too short, the obtained products can also have undesirable characteristics, for example with respect to carbon chain length, content of branched hydrocarbons, content of aromatics, etc. Therefore, in certain embodiments it can be desirable if the residence time of the pyrolysis gas at a temperature above 370° C. is more than 2 seconds, preferably more than 5 seconds, even more preferably more than 10 seconds, such as more than 15 or even more than 20 seconds.

Fast removal of the pyrolysis gas allows high pyrolysis temperatures for cracking the plastic. This has the further advantage that conversion of the plastic can be high and undesired residues are low. For example, the temperature at which at least a portion of the plastic is converted to waxes is at least 370° C., preferably at least 400° C., more preferably at least 415° C., even more preferably at least 425° C. The temperature at which the plastic is converted can be as high as desired, for example up to 850° C., preferably up to 700° C., more preferably up to 600° C., even more preferably up to 500° C., such as up to 480° C. or up to 470° C. In preferred embodiments the temperature at which the plastic is converted ranges from 400 to 650° C., preferably from 425 to 550° C., more preferably from 425 to 520° C., even more preferable 440 to 470° C. Most preferably the temperature at which the plastic is converted ranges from 400 to less than 500° C.

The required low residence time of the pyrolysis gas at a temperature of above 370° C. can be obtained by any suitable means, such as reducing the residence time of the pyrolysis gas in the reactor by operating the reactor under vacuum, by dilution of the pyrolysis gas in the reactor itself, by design of the reactor for example by limiting the volume of the gas phase, or by increasing the percentage of the reactor volume filled by liquid and solid, or by a combination of these measures. Generally, it is preferred to combine several of these measures in order to obtain the desired low residence time.

In one embodiment the reactor is operated at a pressure of less than or equal to 1200 mbar, preferably less than or equal to 1000 mbar, more preferably less than or equal to 950 mbar and even more preferably less than or equal to 900 mbar. The pressure in the reactor can be as low as 0.5 mbar, preferably 1 mbar, preferably 10 mbar, preferably 40 mbar, preferably 50 mbar, more preferably 60 mbar and even more preferably 80 mbar. For example the reactor can be operated at a pressure in the range of 0.5 to 1200 mbar, preferably 10 to 1100 mbar, more preferably 60 to 950 mbar and even more preferably 80 to 900 mbar.

In an alternative or additional embodiment the pyrolysis gas can be diluted with a diluent. Such diluent is not particularly limited but should not adversely affect the pyrolysis reaction or the desired reaction products. In particular, the diluent should have a low oxygen (O₂) content as described below. Examples of suitable diluents are nitrogen, hydrogen, steam, carbon dioxide, combustion gas, hydrocarbon gas and mixtures thereof. The hydrocarbon gas preferably comprises one or more hydrocarbons having less than 5 carbon atoms. Nitrogen, carbon dioxide, combustion gas and hydrocarbon gas with less than 5 carbon atoms are preferred. Combustion gas and hydrocarbon gas with less than 5 carbon atoms are particularly preferred.

The diluent preferably has a low oxygen (O₂) content, such as less than 4% vol, preferably less than 2% vol, more preferably less than 1% vol, each based on the volume of the dry gas. Combustion gas with less than 0.1% vol oxygen based on the dry gas gives particularly good results.

The dilution level is not particularly limited and can be selected according to the requirements. For example, the molar ratio of diluent to pyrolysis product in the pyrolysis gas can be above 0.5, preferably above 0.7, more preferably above 0.8, and even more preferably above 1. A molar ratio of diluent to pyrolysis products in the pyrolysis gas of above 50 is less preferred. Advantageously, this ratio is up to 40, more preferably up to 20. Preferred molar ratios of diluent to pyrolysis products in the pyrolysis gas are in the range of 0.5 to 50, preferably 0.7 to 40 and more preferably 1 to 20, such as 1 to 10 or even 1 to 7.

The diluent may be introduced into the reactor at any position. For example, an inlet for the diluent can be positioned in the top of the reactor so that the diluent basically only comes into contact with the pyrolysis gas but not with the plastic melt under reaction conditions. In an alternative embodiment the diluent inlet can be positioned for example in the bottom part of the reactor so that the diluent comes into contact with the plastic melt under reaction conditions. A combination of two or more different inlets may be used. Preferably at least one diluent inlet is positioned in the bottom part of the reactor. It has been found that in this case the pyrolysis gas is effectively removed from the hot plastic melt thereby effectively reducing the residence time of the pyrolysis gas at a temperature above 370° C. Most preferably a combination of a diluent inlet positioned in the top of the reactor and a diluent inlet positioned in the bottom part of the reactor is employed.

In a particularly preferred embodiment the reactor is operated at a reduced pressure and diluent is used. In this case the molar ratio D of diluent to pyrolysis products in the pyrolysis gas and the absolute pressure P at which the reactor is operated can be adjusted such that D/P is in the range of 2 to 50 mol/mol/bar, particularly in the range of 3 to 30 mol/mol/bar, more particularly in the range of 5 to 20 mol/mol/bar.

The process of the present invention is also characterized in that the weight ratio of catalyst to plastic introduced within the reactor is at least 0.1:30. In preferred embodiments the weight ratio of catalyst to plastic introduced within the reactor is at least 1:30, preferably 2:30, more preferably 3:30, even more preferably at least 4:30, such as at least 5:30.

It has furthermore been surprisingly found that while increasing the ratio of catalyst to plastic introduced in the reactor results in an increase in the overall conversion of the plastic, there is a certain maximum ratio above which the selectivity for waxes is undesirably decreasing. Thus, it was found that there are two contradicting effects of the catalyst. The present invention provides an optimization of these two contradicting effects by also providing an upper limit of the weight ratio of catalyst to plastic introduced within the reactor in case that a high selectivity for waxes is desired. In such case it is preferred that the weight ratio of catalyst to plastic introduced within the reactor is less than or equal to 7:1, preferably less than or equal to 6:1, more preferably less than or equal to 5.5:1.

If both, a high overall conversion and a high selectivity for waxes is desired, the weight ratio of catalyst to plastic introduced within the reactor may for example be in the range of 2:30 to 12:30, more preferably in the range of 5:30 to 10:30.

The catalyst used in the process of the present invention can be any suitable catalyst. Preferred catalysts are those used in FCC operations such as fresh FCC catalyst, spent FCC catalyst, equilibrated FCC catalyst, BCA (bottom cracking additives) or any mixture thereof.

For example, the catalyst can comprise a zeolite-type catalyst. Such catalysts may be selected from crystalline microporous zeolites which are known to the person skilled in the art and which are commercially available. Preferred examples for zeolite-type catalysts are described in WO 2010/135273, the content of which is incorporated herein by reference. Specific examples for suitable zeolite-type catalysts include but are not limited to ZSM-5, ZSM-11, ZSM-22, ZSM-23, ZSM-35, ZSM-48, ZSM-50, TS-1, TS-2, SSZ-46, MCM-22, MCM-49, FU-9, PSH-3, ITQ-1, EU-1, NU-10, silicalite-1, silicalite-2, boralite-C, boralite-D, BCA, and mixtures thereof. Alternatively or additionally, the catalyst may comprise an amorphous-type catalyst which may comprise for example silica, alumina, kaolin, or any mixture thereof. Silica, in particular in the form of sand, is well known for FCC catalyst applications.

In one embodiment of the process of the present invention the conversion of at least a portion of the plastic to waxes is conducted in the presence of a heat carrier. Examples of suitable heat carriers are sand (such as silica), stone, gravel, metal, metal oxides, glass, ceramic, etc. Any metal having a melting point above the temperature at which cracking of the plastic is conducted can be employed. Suitable metals are for example iron and steel, such as forged steel and refractory steel. Sand, metal, such as steel or iron, gravel, and glass are preferred. Sand and steel are particularly preferred.

The heat carrier may be the catalyst for the cracking of the plastic. In a preferred embodiment the heat carrier is, however, not a catalyst for gas-phase cracking of hydrocarbons.

Preferably, the heat carrier or catalyst has a particle size higher than the particle size of the filler used in the plastic.

In one embodiment the heat carrier or catalyst comprises particles, preferably free-flowing particles, for instance granular round particles, near spherical particles, full balls, hollow balls, and the like. Preferably, the heat carrier particles or catalyst particles have a higher particle size than standard US mesh 632, preferably higher than standard US mesh 400. Also preferably the heat carrier particles or catalyst particles have a particle size lower or equal to about 5 cm, preferably lower or equal to about 2.5 cm.

When the heat carrier or catalyst is sand, the particles are advantageously fine or medium sand according to ISO 14688-1:2002. Preferably the heat carrier particles or catalyst particles are fine sand.

When the heat carrier or catalyst is metal, such as iron or steel, they are preferably in the form of full balls. The particle size can be between 1 and 50 mm, preferably between 10 and 30 mm. When the heat carrier or catalyst is glass, the particles are advantageously glass bead or glass balls having a size of between 0.5 and 20 mm, preferably of between 0.6 and 6 mm.

When the heat carrier or catalyst is gravel, it is preferably fine or medium gravel according to ISO 14688-1:2002, preferably fine gravel.

The residence time of the pyrolysis gas in the reactor is expressed as the gas hold-up of the reactor (in other words, the volume of the reactor occupied by the gaseous material and expressed in m³) divided by the flow of gas exiting the reactor and expressed in m³/min. To take into account thermal gas expansion, gases are considered to be at the same temperature of the pyrolysis reactor. If used, gas exiting the reactor comprise the diluent.

The residence time of the condensed material in the reactor is expressed as the condensed material hold-up of the reactor (in other words, the volume of the reactor occupied by the condensed material and expressed in m³) divided by the outlet condensed material flow expressed in m³/min. Temperature expansion of condensed material is neglected. This residence time is not particularly limited but usually is in the range of 1 to 600 minutes, preferably in the range of 2 to 400 minutes, more preferably in the range of 3 to 250 minutes. By “condensed material” the total amount of unconverted raw material in the liquid or solid form, liquid, catalyst and solid products obtained from the reactions (such as for example coke, ashes . . . ) and, if used, the heat carrier is understood.

The process of the present invention can be conducted batchwise or continuously. Conducting the process continuously is preferred.

The skilled person is aware of suitable apparatus and equipment for carrying out the process in accordance with the present invention and he will select the suitable system based on his professional experience, so that no further extensive details need to be given here.

Examples of suitable reactor types are fluidized bed, entrained bed, spouted bed, downcomer, fixed bed, rotating drum, rotating cone, screw cone, screw auger, extruder, molecular distillation, thin film evaporator, kneader, cyclone and the like. Fluidized bed, entrained bed, spouted bed, screw auger and rotating drum are preferred. Screw auger and rotating drum are particularly preferred. Rotating drum gives good results.

Gas exiting the pyrolysis reactor may be cleaned from dust in any de-dusting device. Examples of de-dusting devices are cyclone, multi-cyclone, helical separator, grid separator, swirl tube, electrostatic filter, settling chamber, scroll collector, shutter collector, wet washer and the like. Cyclone, multi-cyclone, helical separator and swirl tube are preferred. Multi-cyclone is particularly preferred.

Separation of the incondensable gas is realized by any ways known by a person skilled in the art. Incondensable gas are meant here as component not condensed at the operating pressure at a temperature of 25° C. Examples of separation devices are quench, organic quench, aqueous quench, spray column, fractionation column, cyclone and the like. Organic quench is preferred. Organic quench operated at a temperature between 110 and 250° C. is preferred, between 125 and 220° C. being particularly preferred. Between 140 and 180° C. being even more preferred.

Vacuum can be provided by any device known by the man skilled in the art. Examples of vacuum devices are liquid ring pump, dry vacuum pump, steam ejector, gas ejector, water ejector and any combination.

Combustion is made in any device known by the man skilled in the art.

Separation of the waxes from the fuel is made by any method known by the man skilled in the art. Examples are evaporation, distillation, crystallization, liquid extraction or a combination. Combination of evaporation and solvent extraction gives good results. Example of solvent are hexane, benzene, toluene, methylethylketone (MEK), methylisobutylketone (MIBK). MEK and MIBK are preferred.

The condensate material separate at the outlet of the reactor may be extracted by any means known by the man skilled in the art. Examples of means are screw, rotating valve and the like. Screw is preferred.

In order to avoid spontaneous ignition, the condensated material may be extracted in an atmosphere with low oxygen content. Less than 2% O₂ in the gas phase surrounding the condensed material is preferred. The condensate material may be cooled down by any means known to the man skilled in the art. Examples are double wall screw conveyor, screw conveyor with water injection, extruder, screw auger and the like. Screw conveyor with water injection is preferred.

Optionally, the condensed material may be sent to a burner to burn the combustible unconverted raw material and the coke. Particularly in the case of having a heat carrier. Optionally, the ashes and the heat carrier are heated in a furnace at a temperature between 500 and 1000° C., preferably between 600 and 800° C. Optionally, at least a portion of the hot ashes and heat carrier (catalyst) is sent to the pyrolysis reactor. Preferably, at least a portion of the ashes are separated from the heat carrier. The separation is made by any method known by the man skilled in the art. Examples of methods are cycloning, elutriation, screening, sieving, centrifuging and the like. The ratio of the heat carrier flow to the raw material flow is usually comprised 0.1 and 10 in weight. Preferably between 0.2 and 8. A ratio higher than 0.25 gives particularly good results.

The process of the present invention yields high-value waxes and mixtures of hydrocarbons comprising predominantly these waxes to be used for example in applications such as candles, adhesives, packaging, rubber, cosmetics, fire logs, bituminous mixtures, superficial wear coatings, asphalt, sealing coatings, etc. The waxes and mixtures of hydrocarbons obtained in the invention have the advantage of having a rather high chain length, in particular a branched carbon chain.

The present invention therefore also relates to a mixture of hydrocarbons, characterized in that the hydrocarbons exhibit a cumulative distribution of their number of carbon atoms such that 20≤d20 and 50≥d50;

≥50 mol % of the hydrocarbons are branched hydrocarbons;

and ≤20 mol % of the unsaturated hydrocarbons are alpha-olefins.

The mixture of hydrocarbons according to the invention predominantly comprises waxes, i.e. hydrocarbons having 20 or more carbon atoms. The mixture may, however, also contain a small amount of hydrocarbons having less than 20 carbon atoms. Preferably, the mixture comprises less than 5 mol %, more preferably less than 3 mol %, even more preferably less than 2 mol % and most preferably less than 1 mol % of hydrocarbons having less than 20 carbon atoms. Here and anywhere else throughout this application “mol % of the hydrocarbons” refers to the total amount of hydrocarbons in the mixture of hydrocarbons.

In preferred embodiments, the hydrocarbons in the mixture of hydrocarbons exhibit a cumulative distribution of their number of carbon atoms such that 22≤d20, preferably 25≤d20.

In a further embodiment, the hydrocarbons in the mixture of hydrocarbons exhibit a cumulative distribution of their number of carbon atoms such that d20≤40, more preferably d20≤35, even more preferably d20≤30.

In a further embodiment, the hydrocarbons in the mixture of hydrocarbons exhibit a cumulative distribution of their number of carbon atoms such that 20≤d20≤40, preferably 22≤d20≤35, more preferably 25≤d20≤30.

In a further embodiment, the hydrocarbons in the mixture of hydrocarbons exhibit a cumulative distribution of their number of carbon atoms such that 45≥d50, preferably 40≥d50.

In a further embodiment, the hydrocarbons in the mixture of hydrocarbons exhibit a cumulative distribution of their number of carbon atoms such that 50≥d50≥20, preferably 40≥d50≥22.

In preferred embodiments, the hydrocarbons in the mixture of hydrocarbons exhibit a cumulative distribution of their number of carbon atoms such that 20≤d20≤40 and 50≥d50≥20, more preferably 22≤d20≤25 and 40≥d50≥22.

Besides the high number of carbon atoms, the hydrocarbons in the mixture of hydrocarbons according to the invention have the advantage that they comprise a high molar amount of branched hydrocarbons. Preferably, ≥60 mol %, more preferably ≥70 mol % of the total amount of hydrocarbons in the mixture of hydrocarbons are branched hydrocarbons.

A further advantage of the hydrocarbons in the mixture of hydrocarbons according to the invention is that the unsaturated hydrocarbons comprise only a low amount of alpha-olefins. Preferably, ≤15 mol %, more preferably ≤10 mol % of the total amount of the unsaturated hydrocarbons are alpha-olefins.

In a further embodiment, the mixture of hydrocarbons has a iodine number of ≥10, preferably of ≥25, more preferably of ≥40.

In a further embodiment, the mixture of hydrocarbons has a iodine number of ≤150, preferably of ≤100, more preferably of ≤70.

In preferred embodiments, the mixture of hydrocarbons has a iodine number in the range of 1 to 150, more preferably in the range of 25 to 100, and even more preferably in the range of 40 to 70.

A further advantage of the mixture of hydrocarbons according to the invention is that the mixture can have a relatively high drop point of for example >25° C., preferably of >40° C., more preferably of >50° C.

The mixture of hydrocarbons according to the invention can consist of hydrocarbons which do not contain any heteroatoms. However, depending on the plastic from which the mixture of hydrocarbons is produced, it is well possible that at least a portion of the hydrocarbons contains one or more heteroatoms, such as oxygen, sulfur, nitrogen or halogen, such as fluorine, chlorine, bromine or iodine. Other heteroatoms are possible as well.

The mixture of hydrocarbons according to the invention can be obtained by the above described process by catalytic cracking of plastic. If the product stream removed from the reactor is selected such that it contains only hydrocarbons with at least 20 carbon atoms, waxes are obtained. However, in practice and in particular on a technical scale, the product stream will usually contain minor amounts of hydrocarbons having less than 20 carbon atoms. In this case, the above described mixture of hydrocarbons is obtained.

### FIGURES

FIG. 1 schematically shows a first embodiment of the process of the present invention.

FIG. 2 schematically shows a second embodiment of the process of the present invention.

FIG. 3 shows the conversion (expressed as %, y-axis) as function of reaction time (expressed in minutes, x-axis) for different N₂ inlet flow: 150 mL/min (triangular-marked line), 1 L/min (empty-circle-marked line) and 4 L/min (full-circle-marked line).

FIG. 4 shows the waxes yield (expressed as wt %, y-axis) as function of conversion (expressed as %, x-axis) for different N₂ inlet flow: 150 mL/min (triangular-marked line), 1 L/min (empty-circle-marked line) and 4 L/min (full-circle-marked line).

FIG. 5 shows the waxes yield (expressed as %, y-axis) as function of the pyrolysis gas residence time (expressed in seconds, x-axis).

FIG. 6 shows the carbon number distribution of the waxes. The plot shows the weight percentage (wt %, y-axis) as function of carbon chain length (expressed as a number, x-axis) for different N₂ inlet flows: 150 mL/min (triangular-marked line), 1 L/min (empty-circle-marked line) and 4 L/min (full-circle-marked line).

FIG. 7 represents the conversion (expressed as %, y-axis) as function of the reaction time (expressed in minutes, x-axis) for different catalyst/plastic ratio: absence of catalyst (rhombus-marked line), 1/30 (empty-triangular-marked line), 5/30 (full-triangular-marked line), 20/30 (empty-circle-marked line), 10/30 (square-marked line).

FIG. 8 shows the cumulative selectivity (expressed as %, y-axis) of the different reaction products (listed in the x-axis) at different catalyst/plastic ratio. Legend: for each product, starting from left to the right the color of the bars refer to ratios of 20/30, 10/30, 5/30, 1/30.

FIG. 9 shows the carbon number distribution of the waxes. The plot shows the weight percentage (wt %, y-axis) as function of carbon chain length (expressed as a number, x-axis) for different catalyst/plastic ratio: 20/30° C. (square-marked line), 10/30 (triangular-marked line), 5/30 (empty-circle-marked line) and 1/30 (full-rhombus-marked line).

FIG. 10 shows the time to reach 65% conversion of plastic (expressed in minutes, y-axis) as function of catalyst initial concentration (expressed as wt %, x-axis).

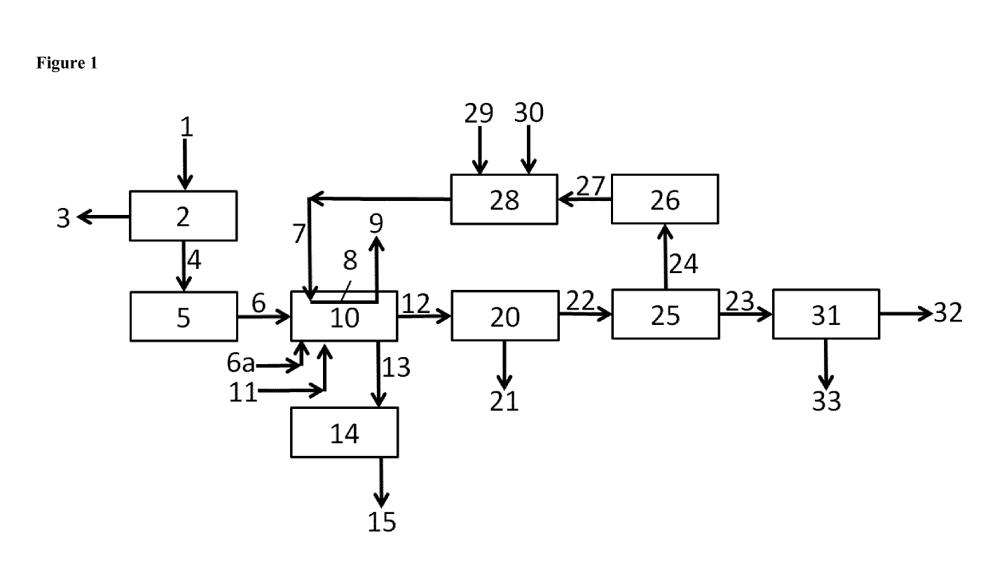

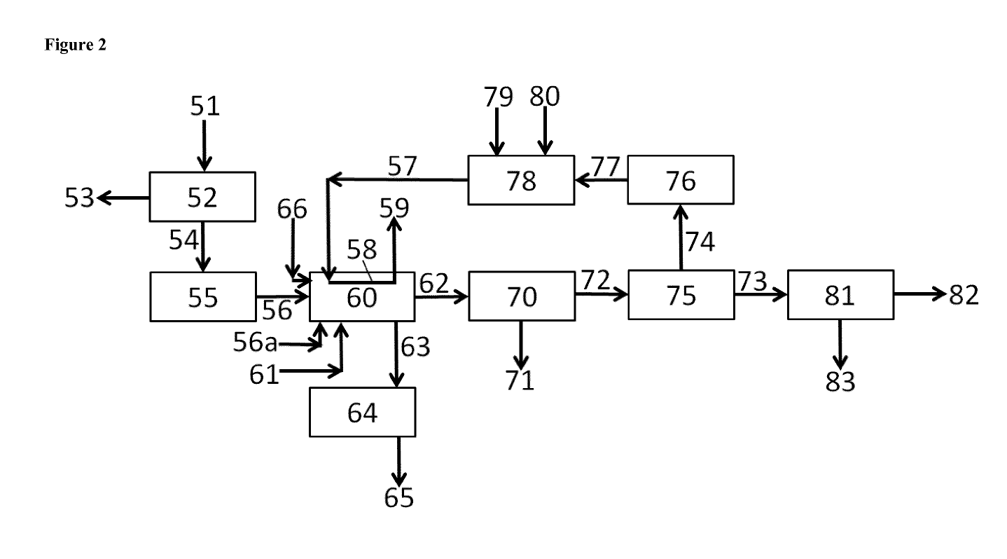

The process of the invention will now be illustrated by way of example with reference to FIGS. 1 and 2.

In a first embodiment, schematically presented in FIG. 1, the pyrolysis of the raw material to produce waxes is realized under vacuum in an oxygen depleted atmosphere. The raw material 1 is pretreated by a combination of physico-chemical treatment 2 that separates an effluent stream 3 and the pretreated raw material 4. The pretreated raw material 4 is introduced with the help of the feeding device 5 in the pyrolysis reactor 10 through the line 6. The pyrolysis reactor is indirectly heated. Without limiting the scope of the present invention, as example the reactor can be heated by the circulation of a hot stream 7 fed to a suitable heat transfer device 8 and recovered at the outlet as the stream 9. Optionally, a heat carrier stream 11 is introduced in the pyrolysis reactor. The pyrolysis gas 12 is recovered from the pyrolysis reactor and sent to a physic-chemical treatment 20. The residue 13 is recovered through the device 14 where it is treated in an adequate way to produce the stream 15. The residue contains the unconverted raw material, by product and optionally the heat carrier introduced in the pyrolysis reactor through the stream 11. In the physico-chemical treatment the pyrolysis gas is cleaned from dust and other detrimental components recovered in stream 21 and separated as a stream 22 that is sent to the treatment 25 where a incondensable stream 24 is separated from the condensate stream 23. The stream 24 is sent to a vacuum device 26. The effluent 27 from the vacuum device is sent to the combustion chamber 28 together with an adequate quantity of combustion air (29) to produce a hot stream 7. Optionally, an auxiliary fuel 30 is added to the combustion chamber 28. The condensate stream 23 is sent to the separation unit 31 where the waxes stream 32 is separated from the by-products 33.

In a second embodiment, the pyrolysis of the raw material to produce waxes realized under vacuum in the presence of a diluent gas is as an example schematically shown in FIG. 2. The pyrolysis of the raw material to produce waxes is realized under vacuum in an oxygen depleted atmosphere. The raw material 51 is pretreated by a combination of physico-chemical treatment 52 that separates an effluent stream 53 and the pretreated raw material 54. The pretreated raw material 54 is introduced with the help of the feeding device 55 in the pyrolysis reactor 60 through the line 56. The pyrolysis reactor is indirectly heated. Without limiting the scope of the present invention, as example the reactor can be heated by the circulation of a hot stream 57 fed to a suitable heat transfer device 58 and recovered at the outlet as the stream 59. Optionally, a heat carrier stream 61 is introduced in the pyrolysis reactor. A gaseous diluent 66 is introduced at a controlled rate in the reactor 60. The pyrolysis gas in mixture with the diluent 62 is recovered from the pyrolysis reactor and sent to a physic-chemical treatment 70. The residue optionally in mixture with the heat carrier 63 is recovered through the device 64 where it is treated in an adequate way to produce the stream 65. The residue contains the unconverted raw material, by product and optionally the heat carrier introduced in the pyrolysis reactor through the stream 61. In the physico-chemical treatment the pyrolysis gas is cleaned from dust and other detrimental components recovered in stream 71 and separated as a stream 72 that is sent to the treatment 75 where a incondensable stream 74 is separated from the condensate stream 73. The stream 74 is sent to a vacuum device 76. The effluent 77 from the vacuum device is sent to the combustion chamber 78 together with an adequate quantity of combustion air (79) to produce a hot stream 57. Optionally, an auxiliary fuel 80 is added to the combustion chamber 78. The condensate stream 73 is sent to the separation unit 81 where the waxes stream 82 is separated from the by-products 83.

Should the disclosure of any patents, patent applications, and publications which are incorporated herein by reference conflict with the description of the present application to the extent that it may render a term unclear, the present description shall take precedence.

### EXAMPLES

### General Description of the Experimental Procedure

In each catalytic run in semi-batch mode, 30 g of plastic (20% polypropylene, 80% polyethylene) were loaded inside the reactor and a defined amount of catalyst (approximately 20 g) is stored in the catalyst storage tank. The reactor was closed and heated from room temperature to 200° C. during 20 minutes, while simultaneously purging with a 150 mL/min nitrogen flow, which was introduced at the top of the reactor vessel. When the internal temperature reached the melting point of the plastic, stirring was started and slowly increased to 690 rpm. The temperature was held at 200° C. for 25-30 minutes. During this heating process, nitrogen coming out from the reactor was not collected. Meanwhile, the catalyst storage tank containing the catalyst was purged with nitrogen several times.

After this first pretreatment step, temperature was increased to the reaction temperature at a heating rate of 10° C./min, and the collection of gases and nitrogen in the corresponding gas sampling bag was started. When the internal temperature reached the reaction temperature, the catalyst was introduced inside the reactor. Depending on the experiments, the nitrogen gas flow was set to 150 mL/min or adjusted to defined values, and the circulation of the gaseous products was commuted to another pair of glass traps and corresponding gas sampling bag. This was considered as the zero reaction time. Depending on the experiments, nitrogen can enter the reactor in two ways: at the top of the vessel or bubbled through the melted plastic.

During selected time periods, liquid and gaseous products were collected in a pair of glass traps and their associated gas sampling bag, respectively. At the end of the experiment the reactor was cooled to room temperature. During this cooling step, liquids and gases were also collected.

The reaction products were classified into 3 groups: i) gases, ii) liquid hydrocarbons and iii) residue (waxy compounds, ashes and coke accumulated on the catalyst). Quantification of the gases was done by gas chromatography (GC) using nitrogen as the internal standard, while quantification of liquids and residue was done by weight. Glass traps (along with their corresponding caps) were weighed before and after the collection of liquids, while the reactor vessel was weighed before and after each run.

The simulated distillation (SIM-DIS) GC method allowed determination of the different fractions in the liquid samples (according to the selected cuts), the detailed hydrocarbon analysis (DHA) GC method allowed determination of the PIONAU components in the gasoline fraction of the last withdrawn sample (C5-C11: Boiling point <216.1° C.; what includes C5-C6 in the gas sample and C5-C11 in the liquid samples), and GC×GC allowed the determination of saturates, mono-, di- and tri-aromatics in the diesel fraction of the last withdrawn liquid samples (C12-C21; 216.1<BP<359° C.).

In all experimental cases, the residence time of the pyrolysis gas was calculated using a reactor volume of 300 mL, a raw plastic density of 0.94 g/mL, a density of the silica of 1.1 g/mL. This leads to a gas hold-up of 250 mL. In the examples, HCO refers to heavy cycle oil which is considered as hydrocarbon molecules with at least 22 carbon atoms (+C22). Waxes refer to hydrocarbon molecules with at least 20 carbon atoms (+C20). In general:
Gasolines: contains C5s and C6s in gases+liquids with bp (boiling point)<150° C. (ca. C5-C9)
Kerosene: liquids with boiling point 150<bp<250° C. (ca. C10-C14)
Diesel: liquids with boiling point 250<bp<359° C. (ca. C15-C21)
HCO: products with boiling point >359° C. (C22 and +)
Waxes: products with boiling point >330° C. (C20 and +)

Determination of the different fractions is done by gas chromatography by the simulated distillation method and according to the ASTM-D-2887 standard.

### General Description of the Analytical Methods

### Measurement of the Number of Carbon Atoms

The number of carbon atoms and their distribution in a mixture of hydrocarbons is measured using the ASTM-D-2887 method. This method is a GC method for the simulated-distillation of complex hydrocarbon mixtures. The method allows separation of the hydrocarbon molecules in a complex mixture according to their boiling point. The boiling point is then related to the carbon number according to defined cut points. In the present invention, the relationship between boiling point and carbon number as defined in table 1 below is used.

TABLE 1

| Carbon | Boiling |
|---|---|
| number | point (° C.) |
| 10 | 150.8-174.1 |
| 11 | 174.1-195.9 |
| 12 | 195.9-216.3 |
| 13 | 216.3-235.4 |
| 14 | 235.4-253.5 |
| 15 | 253.5-270.6 |
| 16 | 270.6-286.8 |
| 17 | 286.8-302 |
| 18 | 302-317 |
| 19 | 317-330 |
| 20 | 330-342.7 |
| 21 | 342.7-359 |
| 22 | 359-368.5 |
| 23 | 368.5-380 |
| 24 | 380-391.3 |
| 25 | 391.3-401 |
| 26 | 401-412.2 |
| 27 | 412.2-422 |
| 28 | 422-431.6 |
| 29 | 431.6-440.8 |
| 30 | 440.8-449.7 |
| 31 | 449.7-458 |
| 32 | 458-467 |
| 33 | 467-474 |
| 34 | 474-482 |
| 35 | 482-490 |
| 36 | 490-497.1 |
| 37 | 497.1-504.1 |
| 38 | 504.1-510.9 |
| 39 | 510.9-517.5 |
| 40 | 517.5-523.9 |
| 41 | 523.9-530.8 |
| 42 | 530.8-536.1 |
| 43 | 536.1-541.9 |
| 44 | 541.9-547.6 |
| 45 | 547.6-553.1 |
| 46 | 553.1-558.4 |
| 47 | 558.4-563.6 |
| 48 | 563.6-568-7 |
| 49 | 568.7-573.6 |
| 50 | 573.6-578.4 |
| 51 | 578.4-583 |
| 52 | 583-587.6 |
| 53 | 587.6-592 |
| 54 | 592-596.4 |
| 55-80 | 596.4-680 |
| >70 | >647.2 |
| >80 | >680 |

Those fractions having a boiling point below 105.8° C. are defined as hydrocarbons having a carbon number of less than 10. For determining the carbon chain length of the molecules in a given sample, the peaks obtained in the GC are integrated according to the boiling point cuts given in table 1 so that the obtained areas under the curves relate to the relative amount of hydrocarbons having the given carbon number for each boiling point range. Normalization of all peaks to 100% allows calculation of the distribution of the number of carbon atoms within the sample according to standard methods known to the person skilled in the art. The obtained distribution is a weight distribution related to the total weight of the sample.

### Measurement of Branched Hydrocarbons

The amount of branched hydrocarbons in the mixture of hydrocarbons according to the invention is determined according to the ASTM-D-6730 method. Measurements are carried out in a Varian 3900 chromatograph equipped with a FID detector and a 100 m capillary column. The GC is also equipped with a back-flush that only allowed a fraction of sample to enter the column. For determination of the composition of the mixture, the Varian DHA software (detailed hydrocarbon analysis) is used. The obtained peaks are integrated and then compared by the DHA software with its internal database to qualify and quantify the peaks. By this technique, the families of molecules which are quantified (paraffins, isoparaffins, olefins, naphtenes and aromatics) are those with boiling points below 216.1° C. For the present invention, it is assumed that the distribution that is observed in the gasolines having a boiling point below 216.1° C. is identical to that in the hydrocarbons having a higher boiling point.

### Measurement of Unsaturated Hydrocarbons and Alpha-Olefins

The amount of alpha-olefins in the unsaturated hydrocarbons in the mixture according to the invention was determined using usual ¹H and ¹³C NMR techniques. For example, in CDCl₃ as solvent 1-alkenes show a peak at 5.82 ppm, 2-alkenes show a peak at 5.42 ppm and 2-methyl-1-alkenes show a peak at 4.69 ppm. Those unsaturated hydrocarbons which comprise both, alpha-double bonds and other double bonds can be distinguished by the GC method described above for the determination of the branched hydrocarbons. Alpha-olefins which comprise both, alpha-double bonds and other double bonds qualify as alpha-olefins in the context of the present invention.

### Measurement of Iodine Number

The iodine number of the mixture of hydrocarbons according to the invention is measured by dissolving between 0.1 and 0.2 g of the sample in 10 ml of chloroform. 5 ml of Wijs solution comprising 0.1 M ICl are added to the solution and the mixture is allowed to react for 1 hour in the dark. The unreacted Wijs solution is then reacted with a potassium iodide solution at 100 g/l to convert unreacted ICl to I₂. The amount of formed I₂ is determined by titration using a thiosulfate solution. From the amount of thiosulfate required to react with the I₂, the amount of unreacted Wijs solution is calculated indicating the number of unsaturated bonds in the hydrocarbons.

### Measurement of Drop Point

The drop point of the mixture of hydrocarbons according to the invention is measured according to European standard EN1427 of March 2007.

### Example 1

The experiment was carried out following the general procedure described above. Experiments were carried out using 80 wt. % HDPE and 20 wt. % PP as raw materials and 20 g of bottom cracking additive BCA-105 purchased from Johnson Matthey. Reaction temperature was set to 450° C. and N₂ flow varied from 0.15 L/min to 4 L/min. Catalyst to plastic weight ratio was equal to 20/30 by wt.

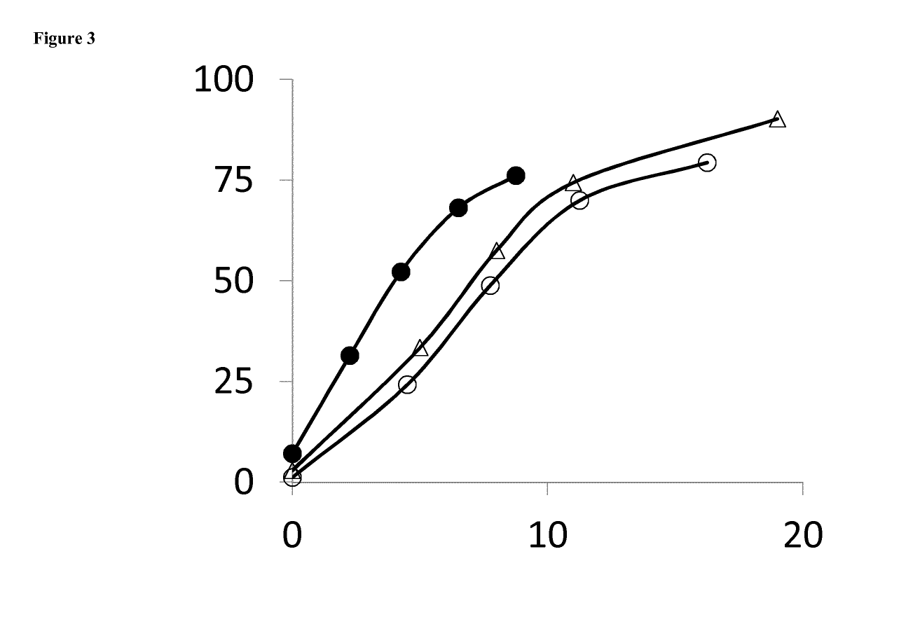

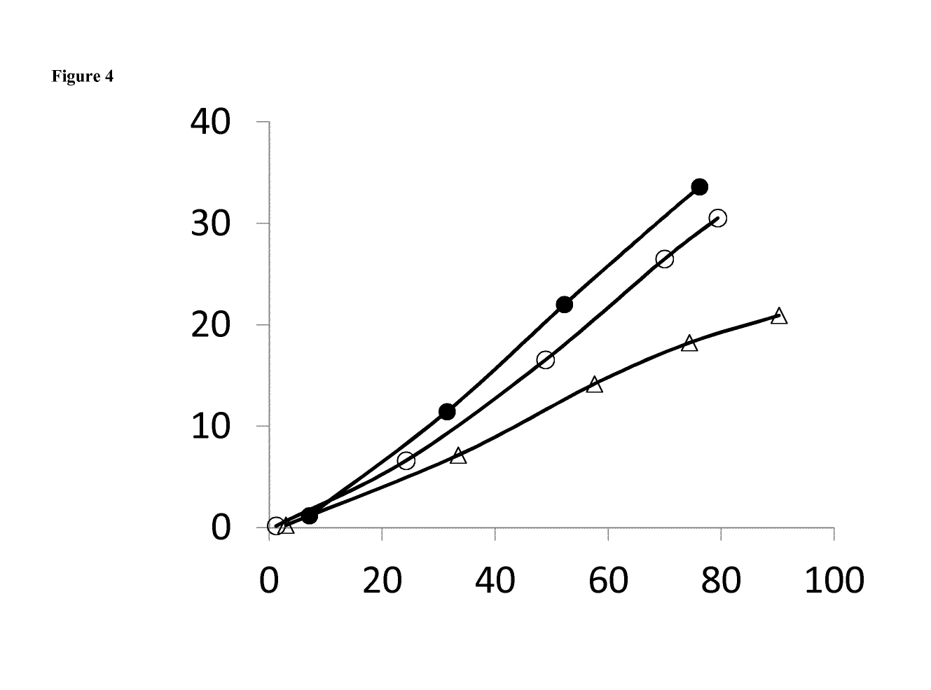

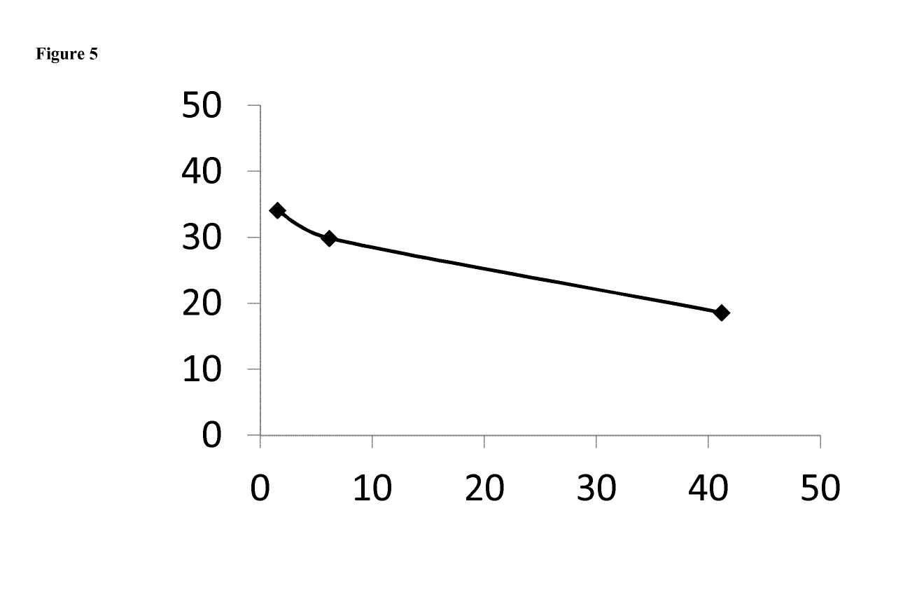

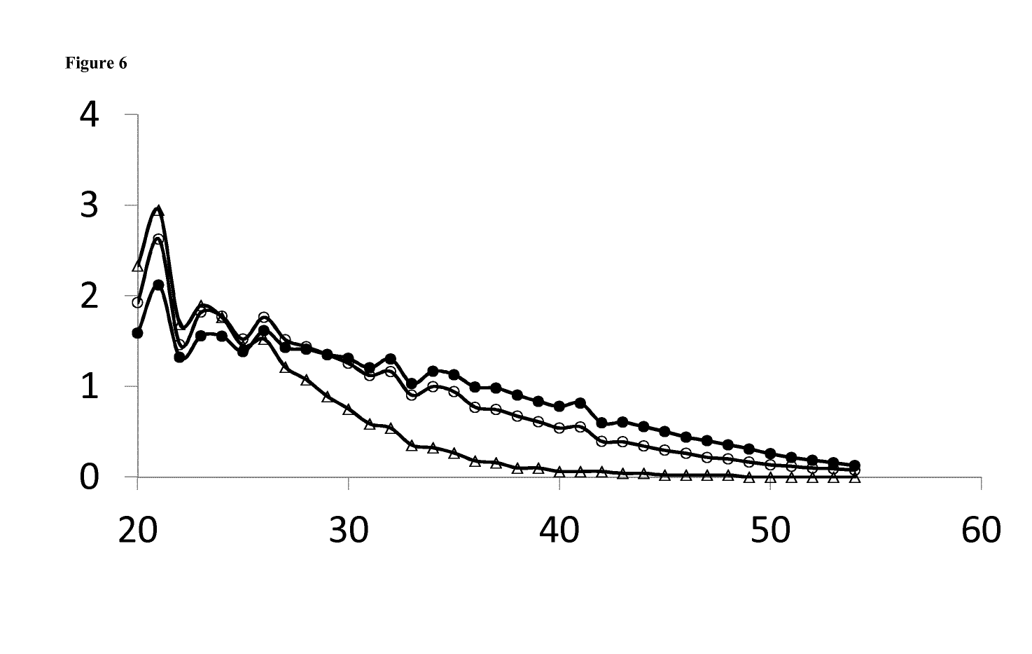

Experimental results are shown in FIGS. 3 to 6. FIG. 3 shows the surprising effect that decreasing the pyrolysis gas residence time by increasing the diluent flow results in an increase in overall conversion and an increase in the yield of waxes (FIG. 4). The dependency between gas phase residence time and yield of waxes is also shown in FIG. 5. The results shown in FIG. 6 demonstrate that decreasing the pyrolysis gas residence time by increasing the diluent flow surprisingly shifts the carbon chain distribution towards longer chain compounds. For a gas flow of 150 mL/min, the hydrocarbon mixture had a cumulative distribution of the number of carbon atoms of d20=20 and d50=23. For a gas flow of 1 L/min, the cumulative distribution of the number of carbon atoms was d20=22 and d50=27. For a gas flow of 4 L/min, the cumulative distribution of carbon atoms was d20=22 and d50=29.

The hydrocarbon mixture obtained using BCA as catalyst was analyzed. The results of this analysis are summarized in the following table 2.

TABLE 2

| | | | | C₃₀-C₄₀ | C₃₀-C₄₀ |
|---|---|---|---|---|---|
| | Crude | <C₂₀ cut | C₂₀-C_{30 cut} | cut up | cut down |
| Melting | 109 | 8 | 30 | 114 | 114 |
| point | | | | | |
| Iodine | 56 | 60 | 64 | 31 | 32 |
| number | | | | | |
| Drop point | 109.0 | ND | ND | 115.5 | 116.0 |
| C, H | 82.1/13.3 | 85.7/14.1 | 85.7/13.9 | 84.8/13.9 | 66.9/11.6 |
| 1-olefins/ | 4.3/95.6 | 4.1/95.9 | 1.8/98.2 | 100 | Pb |
| internal | | | | | |
| olefins | | | | | |
| Aspect | Pasty solid | oily | Pasty solid | solid | solid |

### Example 2

The experiment was carried out following the general procedure described above. Experiments were carried out using 80 wt. % HDPE and 20 wt. % PP as raw materials and 20 g of a zeolite-type catalyst (Equilibrium fluidized catalytic cracking catalyst (labeled ECAT-DC) provided by Equilibrium Catalyst Inc.). Reaction temperature was set to 425° C. and N₂ flow was set to 0.15 L/min. Catalyst to plastic weight ratio was varied from 20:30 to 1:30. A comparative example with no catalyst addition has also been included to highlight the strong effect of catalyst addition on reaction kinetics. The residence time of pyrolysis gas is estimated 40-46 s range.

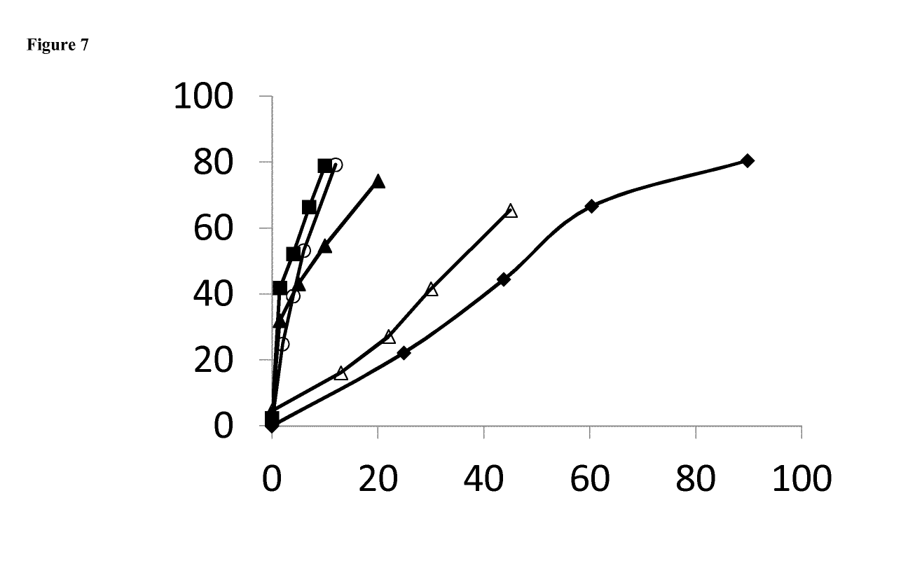

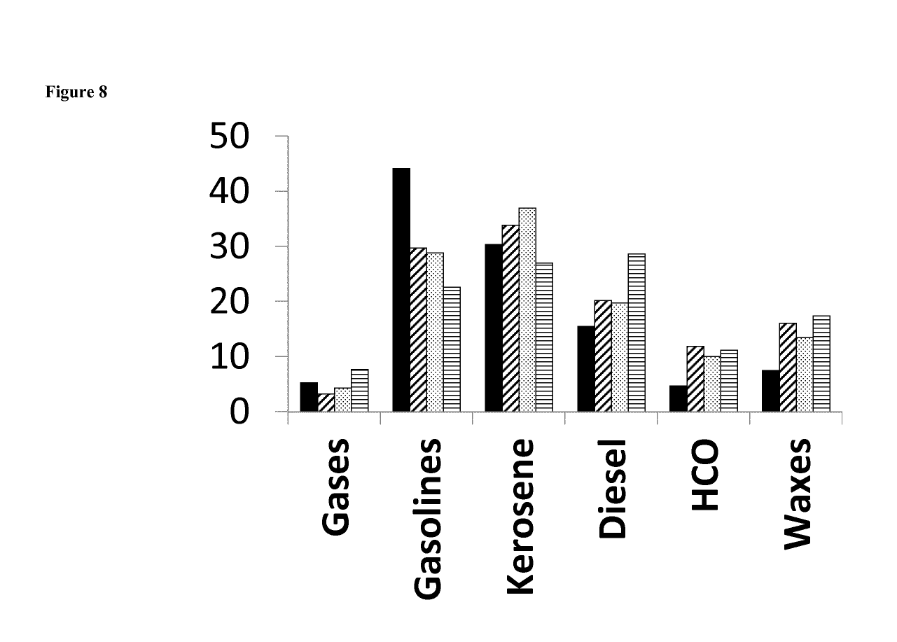

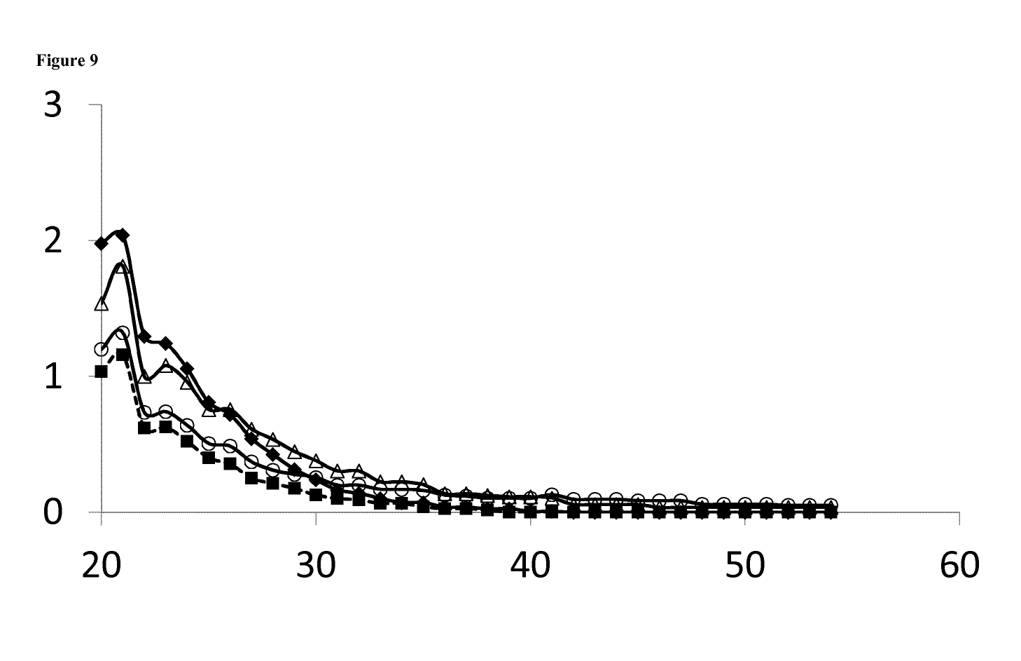

Experimental results are shown in FIGS. 7 to 10. FIG. 7 shows that reaction kinetic is strongly influenced by the amount of catalyst used, particularly a minimum ratio of 5/30 seems preferable to have a significant increase in the conversion rate. On the other hand, HCO yield strongly increases when the ratio is 10/30 or lower (FIG. 8). FIG. 9 shows that quality of waxes obtained with varying quantity of catalyst does not change and roughly the same carbon chain distribution is obtained for all cases. The cumulative distributions of carbon atoms in the obtained mixtures of hydrocarbons depending on the catalyst to plastic weight ratio are summarized in the following table 3.

TABLE 3

Catalyst/plastic

| | 20/30 | 10/30 | 5/30 | 1/30 |
|---|---|---|---|---|
| d20 | 20 | 20 | 20 | 20 |
| d50 | 22 | 23 | 24 | 22 |

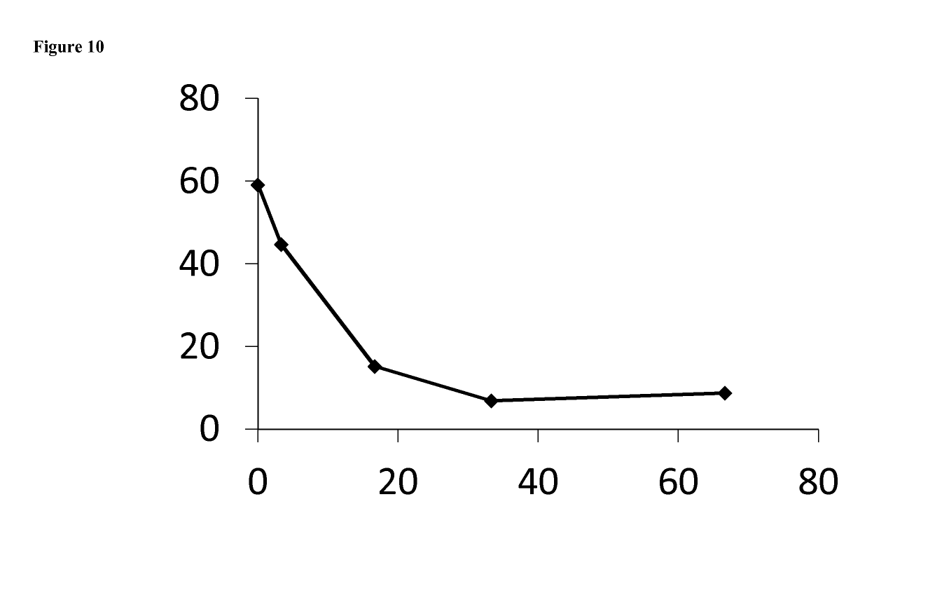

FIG. 10 shows the beneficial effect of using a catalyst on the reaction kinetics. Indeed, same plastic conversion can be achieved in a much shorter time.

### Example 3: Mass Balance According to the Invention

In this and the following example, the post-consumer plastic are named from their main plastic component; on average, the considered post-consumer plastic contain 8% weight of additives.

A unit equipped with a double-wall rotating drum furnace of 1.4 m of internal diameter and 5 m internal length equipped with a gaseous product uptake line of 400 mm of diameter and 2 m long at a temperature above 370° C. was fed with 1500 kg/h of post-consumer mixed plastic waste of the following composition:

| | g/kg |
|---|---|
| PE | 415.2 |
| PP | 400.0 |
| PS | 20.0 |
| PVC | 20.0 |
| Water | 100.0 |
| Food residue | 20.0 |
| Foreign solid | 20.0 |
| Air | 4.8 |

The mixed plastic waste was first pretreated at 250° C. and atmospheric pressure to melt the plastic and remove most of the air, water, food residue and foreign solid as an effluent. This effluent contains also the gaseous product resulting from the decomposition of any component of the fed material. The pretreated material was introduced in the double-wall rotating drum furnace operating at 465° C. under 218 mbar absolute pressure as well as 1500 kg/h of a heat carrier constituted of fine sand and 750 kg of FCC catalyst Johnson Matthey's INTERCAT_{JM} bottoms cracking additive BCA-105 supplied both at 700° C. The gas hold-up in the furnace was estimated to 5.3 m³ and the condensed phase hold-up to 2.4 m³. The supplementary gas hold-up at a temperature equal or above 370° C. is estimated to 0.3 m³. The total gas flow produced flow produced at the outlet of the furnace was estimated to 10.2 kmol/h corresponding to 1189 kg/h.

The residence time of the gaseous products in the gas phase at or above 370° C. was calculated to 8 s. The calculated flows are given in the following table 4 with the numbering of FIG. 1.

TABLE 4

| | Average Molar Mass | 1 kg/h | 3 kg/h | 4 kg/h | 6a kg/h | 11 kg/h | 13 kg/h | 14 kg/h | 23 kg/h | 24 kg/h | 32 kg/h | 33 kg/h |
|---|---|---|---|---|---|---|---|---|---|---|---|---|
| PE | | 623 | | 623 | | | | | | | | |
| PP | | 600 | | 600 | | | | | | | | |
| PS | | 30 | | 30 | | | | | | | | |
| PVC | | 30 | | 30 | | | | | | | | |
| Water | | 150 | 150 | | | | | | | | | |
| Organic impurities | | 30 | 30 | | | | | | | | | |
| Foreign solids | | 30 | 30 | | | | | | | | | |
| Gases | 30 | | | | | | | 136 | | 136 | | |
| Gasoline | 100 | | | | | | | 211 | 211 | | 211 | |
| Kerosene | 130 | | | | | | | 129 | 129 | | 129 | |
| Diesel | 180 | | | | | | | 149 | 149 | | 149 | |
| Waxes | 400 | | | | | | | 516 | 516 | | | 516 |
| Coke | | | | | | | 38 | | | | | |
| Catalyst | | | | | 750 | | 750 | | | | | |
| Ashes | | | | | | | 64 | | | | | |
| Heat carrier | | | | | | 1500 | 1500 | | | | | |
| Air | | 7 | 7 | | | | | | | | | |
| Total | | 1500 | 217 | 1283 | 750 | 1500 | 2353 | 1142 | 1006 | 136 | 490 | 516 |

The heat duty of the reaction was estimated at 449 kW, the heat supplied by the heat carrier and the catalyst to 139 kW corresponding to 47% of the heat available by combustion of the coke and the heat supplied by the double-wall to 310 kW corresponding to 20% of the heat available by combustion of the gases. The heat transfer surface available in the furnace was estimated to 20 m², the overall heat transfer coefficient is estimated to 80 W/m²K and the logarithmic difference of temperature between the hot gases used to heat up the furnace and the reaction medium reached 196° C.

The residence time of the condensed material in the furnace was estimated to 79 min. The waxes overall yield was calculated to 40.2% based on the plastic content (including the additives) of the mixed plastic waste. The waxes were mostly branched.

### Example 4: Mass Balance According to the Invention

A unit equipped with a double-wall rotating drum furnace of 1.4 m of internal diameter and 5 m internal length equipped with a gaseous product uptake line of 400 mm of diameter and 5 m long at a temperature above 370° C. was fed with 950 kg/h of post-consumer mixed plastic waste of the following composition:

| | g/kg |
|---|---|
| PE | 415.2 |
| PP | 400.0 |
| PS | 20.0 |
| PVC | 20.0 |
| Water | 100.0 |
| Food residue | 20.0 |
| Foreign solid | 20.0 |
| Air | 4.8 |

The mixed plastic waste was first pretreated at 250° C. and atmospheric pressure to melt the plastic and remove most of the air, water, food residue and foreign solid as an effluent. This effluent contained also the gaseous product resulting from the decomposition of any component of the fed material. The pretreated material was introduced in the double-wall rotating drum furnace operating at 465° C. under 350 mbar absolute pressure as well as 950 kg/h of a heat carrier constitute of 20 mm diameter refractory steel ball and 750 kg of FCC catalyst Johnson Matthey's INTERCAT_{JM} bottoms cracking additive BCA-105 supplied both at 700° C. and 560 kg/h of nitrogen at 25° C. The gas hold-up in the furnace was estimated to 6.6 m³ and the condensed phase hold-up to 1.1 m³. The supplementary gas hold-up at a temperature equal or above 370° C. is estimated to 0.3 m³. The total gas flow produced at the outlet of the furnace is estimated to 26.2 kmol/h corresponding to 1283 kg/h.

The residence time of the gaseous products in the gas phase at or above 370° C. was calculated to 5.9 s. The calculated flows are given in the following table 5 with the numbering of FIG. 2.

TABLE 5

| | Average | 51 | 53 | 64 | 56a | 61 | | | | | | | |
|---|---|---|---|---|---|---|---|---|---|---|---|---|---|
| | Molar Mass | kg/h | kg/h | kg/h | kg/h | kg/h | 63 kg/h | 62 kg/h | 66 kg/h | 73 kg/h | 74 kg/h | 32 kg/h | 33 kg/h |
| PE | | 394 | | 394 | | | | | | | | | |
| PP | | 380 | | 380 | | | | | | | | | |
| PS | | 19 | | 19 | | | | | | | | | |
| PVC | | 19 | | 19 | | | | | | | | | |
| Water | | 95 | 95 | | | | | | | | | | |
| Organic impurities | | 19 | 19 | | | | | | | | | | |
| Foreign solids | | 19 | 19 | | | | | | | | | | |
| Gases | 30 | | | | | | | 86 | | | 86 | | |
| Gasoline | 100 | | | | | | | 134 | | 134 | | 134 | |
| Kerosene | 130 | | | | | | | 81 | | 81 | | 81 | |
| Diesel | 180 | | | | | | | 95 | | 95 | | 95 | |
| Waxes | 400 | | | | | | | 328 | | 328 | | | 328 |
| Coke | | | | | | | 24 | | | | | | |
| Catalyst | | | | | 475 | | 475 | | | | | | |
| Ashes | | | | | | | 41 | | | | | | |
| Heat carrier | | | | | | 950 | 950 | | | | | | |
| Nitrogen | | | | | | | | 560 | 560 | | 560 | | |
| Air | | 5 | 5 | | | | | | | | | | |
| Total | | 950 | 138 | 812 | 475 | 950 | 1490 | 1283 | 560 | 637 | 646 | 309 | 328 |

The heat duty of the reaction, including the preheating of the nitrogen was estimated at 341 kW, the heat supplied by the heat carrier to 98 kW corresponding to 49% of the heat available by combustion of the coke and the heat supplied by the double-wall to 243 kW corresponding to 16% of the heat available by combustion of the gases. The heat transfer surface available in the furnace was estimated to 20 m², the overall heat transfer coefficient was estimated to 80 W/m²K and the logarithmic difference of temperature between the hot gases used to heat up the furnace and the reaction medium reach 153° C.

The residence time of the condensed material in the furnace is estimated to 93 min. The specific dilution ratio D/P is calculated to 9.3 mol/mol/bar. The waxes overall yield is calculated to 40.3% based on the plastic content (including the additives) of the mixed plastic waste. The waxes were mostly branched.

### Example 5: Mass Balance According to the Invention

A unit equipped with a double-wall rotating drum furnace of 1.4 m of internal diameter and 5 m internal length equipped with a gaseous product uptake line of 400 mm of diameter and 5 m long at a temperature above 370° C. was fed with 700 kg/h of post-consumer mixed plastic waste of the following composition:

| | g/kg |
|---|---|
| PE | 415.2 |
| PP | 400.0 |
| PS | 20.0 |
| PVC | 20.0 |
| Water | 100.0 |
| Food residue | 20.0 |
| Foreign solid | 20.0 |
| Air | 4.8 |

The mixed plastic waste is first pretreated at 250° C. and atmospheric pressure to melt the plastic and remove most of the air, water, food residue and foreign solid as an effluent. This effluent contains also the gaseous product resulting from the decomposition of any component of the fed material. The pretreated material was introduced in the double-wall rotating drum furnace operating at 465° C. under 155 mbar absolute pressure as well as 350 kg/h of FCC catalyst Johnson Matthey's INTERCAT_{JM} bottoms cracking additive BCA-105 supplied at 25° C. The gas hold-up in the furnace was estimated to 6.9 m³ and the condensed phase hold-up to 0.7 m³. The supplementary gas hold-up at a temperature equal or above 370° C. was estimated to 0.3 m³. The total gas flow produced at the outlet of the furnace is estimated to 5.8 kmol/h corresponding to 679 kg/h.

The residence time of the gaseous products in the gas phase at or above 370° C. was calculated to 15.9 s. The calculated flows are given in the following table 6 with the numbering of the FIG. 1.

TABLE 6

| | Average | | | | | | | | | | |
|---|---|---|---|---|---|---|---|---|---|---|---|
| | Molar Mass | 1 kg/h | 3 kg/h | 4 kg/h | 6a kg/h | 13 kg/h | 14 kg/h | 23 kg/h | 24 kg/h | 32 kg/h | 33 kg/h |
| PE | | 291 | | 291 | | | | | | | |
| PP | | 280 | | 280 | | | | | | | |
| PS | | 14 | | 14 | | | | | | | |
| PVC | | 14 | | 14 | | | | | | | |
| Water | | 70 | 70 | | | | | | | | |
| Organic impurities | | 14 | 14 | | | | | | | | |
| Foreign solids | | 14 | 14 | | | | | | | | |
| Gases | 30 | | | | | | 63 | | 63 | | |
| Gasoline | 100 | | | | | | 98 | 98 | | 98 | |
| Kerosene | 130 | | | | | | 63 | 63 | | 63 | |
| Diesel | 180 | | | | | | 70 | 70 | | 70 | |
| Waxes | 400 | | | | | | 238 | 238 | | | 238 |
| Coke | | | | | | 18 | | | | | |
| Catalyst | | | | | 350 | 350 | | | | | |
| Ashes | | | | | | 30 | | | | | |
| Air | | 3 | 3 | | | | | | | | |
| Total | | 700.0 | 101.4 | 598.6 | 350.0 | 397.9 | 532.8 | 469.7 | 63.1 | 231.6 | 238.1 |

The heat duty of the reaction, including the preheating of the nitrogen and the catalyst was estimated at 307 kW and the heat supplied by the double-wall to 307 kW corresponding to 34% of the heat available by combustion of the gases. The heat transfer surface available in the furnace was estimated to 20 m², the overall heat transfer coefficient is estimated to 80 W/m²K and the logarithmic difference of temperature between the hot gases used to heat up the furnace and the reaction medium reached 194° C.

The residence time of the condensed material in the furnace is estimated to 108 min. The waxes overall yield was calculated to 39.8% based on the plastic content (including the additives) of the mixed plastic waste. The waxes were mostly branched.

### Reference Example 1: Mass Balance for Condition Outside the Invention

A unit equipped with a double-wall rotating drum furnace of 1.4 m of internal diameter and 5 m internal length equipped with a gaseous product uptake line of 400 mm of diameter and 2 m long at a temperature above 370° C. was fed with 1100 kg/h of post-consumer mixed plastic waste of the following composition:

| | g/kg |
|---|---|
| PE | 415.2 |
| PP | 400.0 |
| PS | 20.0 |
| PVC | 20.0 |
| Water | 100.0 |
| Food residue | 20.0 |
| Foreign solid | 20.0 |
| Air | 4.8 |

The mixed plastic waste was first pretreated at 250° C. and atmospheric pressure to melt the plastic and remove most of the air, water, food residue and foreign solid as an effluent. This effluent contained also the gaseous product resulting from the decomposition of any component of the fed material. The pretreated material was introduced in the double-wall rotating drum furnace operating at 465° C. under 1340 mbar absolute pressure as well as 550 kg/h of FCC catalyst Johnson Matthey's INTERCAT_{JM} bottoms cracking additive BCA-105 supplied at 25° C. The gas hold-up in the furnace was estimated to 6.7 m³ and the condensed phase hold-up to 0.7 m³. The supplementary gas hold-up at a temperature equal or above 370° C. was estimated to 0.3 m³. The total gas flow produced flow produced at the outlet of the furnace is estimated to 7.8 kmol/h corresponding to 837 kg/h.

The residence time of the gaseous products in the gas phase at or above 370° C. was calculated to 76 s. The calculated flows are given in the following table 7 with the numbering of FIG. 1.

TABLE 7

| | Average | | | | | | | | | | |
|---|---|---|---|---|---|---|---|---|---|---|---|
| | Molar Mass | 1 kg/h | 3 kg/h | 4 kg/h | 6a kg/h | 13 kg/h | 14 kg/h | 23 kg/h | 24 kg/h | 32 kg/h | 33 kg/h |
| PE | | 457 | | 457 | | | | | | | |
| PP | | 440 | | 440 | | | | | | | |
| PS | | 22 | | 22 | | | | | | | |
| PVC | | 22 | | 22 | | | | | | | |
| Water | | 110 | 110 | | | | | | | | |
| Organic impurities | | 22 | 22 | | | | | | | | |
| Foreign solids | | 22 | 22 | | | | | | | | |
| Gases | 30 | | | | | | 94 | | 94 | | |
| Gasoline | 100 | | | | | | 195 | 195 | | 195 | |
| Kerosene | 130 | | | | | | 175 | 175 | | 175 | |
| Diesel | 180 | | | | | | 140 | 140 | | 140 | |
| Waxes | 400 | | | | | | 233 | 233 | | | 233 |
| Coke | | | | | | 28 | | | | | |
| Catalyst | | | | | 550 | 550 | | | | | |
| Ashes | | | | | | 47 | | | | | |
| Air | | 27 | 27 | | | | | | | | |
| Total | | 1100 | 181 | 941 | 550 | 625 | 837 | 744 | 94 | 510 | 233 |

The heat duty of the reaction, including the preheating of the catalyst was estimated at 334 kW, and the heat supplied by the double-wall to 334 kW corresponding to 40% of the heat available by combustion of the gases. The heat transfer surface available in the furnace was estimated to 20 m², the overall heat transfer coefficient was estimated to 80 W/m²K and the logarithmic difference of temperature between the hot gases used to heat up the furnace and the reaction medium reached 260° C.

The residence time of the condensed material in the furnace was estimated to 110 min. The waxes overall yield was calculated to 24.7% based on the plastic content (including the additives) of the mixed plastic waste. The waxes were mostly branched.

### Reference Example 2: Mass Balance for Condition Outside the Invention

A unit equipped with a double-wall rotating drum furnace of 1.4 m of internal diameter and 5 m internal length equipped with a gaseous product uptake line of 400 mm of diameter and 2 m long at a temperature above 370° C. was fed with 800 kg/h of post-consumer mixed plastic waste of the following composition:

| | g/kg |
|---|---|
| PE | 415.2 |
| PP | 400.0 |
| PS | 20.0 |
| PVC | 20.0 |
| Water | 100.0 |
| Food residue | 20.0 |
| Foreign solid | 20.0 |
| Air | 4.8 |

The mixed plastic waste was first pretreated at 250° C. and atmospheric pressure to melt the plastic and remove most of the air, water, food residue and foreign solid as an effluent. This effluent contains also the gaseous product resulting from the decomposition of any component of the fed material. The pretreated material was introduced in the double-wall rotating drum furnace operating at 465° C. under 1420 mbar absolute pressure as well as 56 kg/h of nitrogen and 400 kg/h of FCC catalyst Johnson Matthey's INTERCAT_{JM} bottoms cracking additive BCA-105 supplied both at 25° C. The gas hold-up in the furnace is estimated to 6.9 m³ and the condensed phase hold-up to 0.8 m³. The supplementary gas hold-up at a temperature equal or above 370° C. is estimated to 0.3 m³. The total gas flow produced at the outlet of the furnace is estimated to 7.8 kmol/h corresponding to 665 kg/h.

The residence time of the gaseous products in the gas phase at or above 370° C. was calculated to 83.6 s. The calculated flows are given in the following table 8 with the numbering of FIG. 2.

TABLE 8

| | Average | 51 | 53 | | | | | | | | | |
|---|---|---|---|---|---|---|---|---|---|---|---|---|
| | Molar Mass | kg/h | kg/h | 64 kg/h | 56a kg/h | 63 kg/h | 62 kg/h | 66 kg/h | 73 kg/h | 74 kg/h | 32 kg/h | 33 kg/h |
| PE | | 332 | | 332 | | | | | | | | |
| PP | | 320 | | 320 | | | | | | | | |
| PS | | 16 | | 16 | | | | | | | | |
| PVC | | 16 | | 16 | | | | | | | | |
| Water | | 80 | 80 | | | | | | | | | |
| Organic impurities | | 16 | 16 | | | | | | | | | |
| Foreign solids | | 16 | 16 | | | | | | | | | |
| Gases | 30 | | | | | | 68 | | | 68 | | |
| Gasoline | 100 | | | | | | 146 | | 146 | | 146 | |
| Kerosene | 130 | | | | | | 130 | | 130 | | 130 | |
| Diesel | 180 | | | | | | 136 | | 136 | | 136 | |
| Waxes | 400 | | | | | | 128 | | 128 | | | 128 |
| Coke | | | | | | 21 | | | | | | |
| Catalyst | | | | | 400 | 400 | | | | | | |
| Ashes | | | | | | 34 | | | | | | |
| Nitrogen | | | | | | | 56 | 56 | | 56 | | |
| Air | | 4 | 4 | | | | | | | | | |
| Total | | 800 | 116 | 684 | 400 | 455 | 665 | 56 | 541 | 124 | 413 | 128 |

The heat duty of the reaction, including the preheating of the nitrogen and the catalyst was estimated at 311 kW, and the heat supplied by the double-wall to 311 kW corresponding to 43% of the heat available by combustion of the gases. The heat transfer surface available in the furnace was estimated to 19 m², the overall heat transfer coefficient is estimated to 80 W/m²K and the logarithmic difference of temperature between the hot gases used to heat up the furnace and the reaction medium reached 196° C.

The residence time of the condensed material in the furnace was estimated to 102 min. The specific dilution ratio D/P is calculated to 0.24 mol/mol/bar. The waxes overall yield is calculated to 18.6% based on the plastic content (including the additives) of the mixed plastic waste. The waxes were mostly branched.

Waxes of the type obtained according to the process of the invention may, inter alia, be used as additives in bituminous coating compositions, and more generally in coating compositions on the basis (i) of mineral aggregates and (ii) of organic binders derived from petroleum (bitumen or mixtures of synthetic polymeric resins and oil) and/or from plants (in particular binders on the basis of resins and plant oils). Waxes of the invention are especially useful in bituminous mixtures and asphalt concretes, based on pure or modified bitumen (in particular through addition of polymers), as well as in coatings based on other organic binders, for example of the type of synthetic polymers and/or plant resins.

In a first aspect, the waxes according to the invention, when used in coatings on the basis of mineral aggregates and organic binders, can be employed to facilitate the use of the binder and/or the mixture of binder and aggregate; and/or to optimize the coating of the aggregates, and particularly in the heat: the presence of waxes according to the invention tends to decrease, typically by several tens of degrees Celsius, the temperature at which the compositions are sufficiently fluid to be used, which manifests itself especially in terms of reduced process costs.

According to another aspect, compatible with the previous and complementary thereto for certain applications of the type exemplified below, the waxes according to the invention may be used in a coating based on mineral aggregates and organic binders to increase hardening speed of the coating during its cooling. The waxes according to the invention indeed tend to have a “setting” speed higher than organic binders such as bitumen or clear binders mentioned above.

Thus, by way of illustration and not limitation, a wax of the invention may be advantageously used at least in the following applications:
In so-called “warm” bituminous mixtures, obtained by coating aggregates with heated bitumen (pure or modified): for this application, the wax is advantageously mixed with the bitumen before the coating of the aggregates, whereby the bitumen can be mixed with aggregates at a much lower temperature than in the absence of wax (typically at a temperature of about 110-140° C. compared to 200° C. in the absence of resin, more precisely, 150-200° C.).
In superficial wear coatings where the wax is typically mixed with a bitumen, a flux additive of the plant oil type, for example as described, inter alia, in EP 1845134. This fluxed binder is intended to be sprayed onto a road surface on which aggregates are then deposited. The presence of waxes allows in this context not only to reduce the temperature at which it is sprayed, but also to increase the speed of cohesion increase (setting) of the bitumen after its deposition and this despite the presence of fluxing agents.
In poured asphalts, namely the compositions of the type of bituminous mixture mentioned above, but having a higher bitumen content (typically at least 10% by weight based on the total weight of the mix, against 4.5 to 5.5% by weight in conventional bituminous mixtures): the wax is typically mixed with the bituminous binder, whereby the bitumen can be mixed with the aggregates at a temperature of about 160 to 190° C., compared to a temperature above 200° C. (typically about 250° C.) in the absence of wax. The wax also imparts curing properties.
In bituminous mixtures based on “clear binders” also called “synthetic binders”, i.e. based on binders on the basis of synthetic polymers and/or resins and oil of petroleum origin and/or of plant origin of the transparent type allows the aggregates they contain to be distinguished, in contrast to a bituminous mixtures: the presence of waxes in these binders allows, again, to reduce the temperature at which the coating is made and deposited.
In sealing coatings, in particular for roofs, which comprise bitumen mixed with polymers: the presence of wax also here allows reducing the temperature at which the coatings are manufactured. It also allows an acceleration of the setting of the coating after deposition, which is particularly appreciable in the case of deposits on pitched roofs where the deposited composition tends to flow if it does not harden sufficiently rapidly.

Moreover, a wax of the invention may be used to improve the rheological properties of binders and more specifically, to increase the modulus of rigidity. A wax of the invention may, in this context, additionally provide lubricating properties.

On the other hand, at least in some cases, the presence of a wax according to the invention in a bituminous coating tends to improve the resistance to embrittlement of the coating in the face of solubilization by hydrocarbons. The waxes according to the invention are found in this context particularly suitable as additives in bituminous coatings that are intended to come into contact with gasoline or kerosene, such as bituminous coatings used in service stations.

1. A process for converting plastic into waxes by catalytic cracking, the process comprising:
introducing the plastic and a catalyst within a reactor;
allowing at least a portion of the plastic to be converted to waxes, the waxes being part of the pyrolysis gas formed within the reactor; and
removing a product stream containing said waxes from the reactor;
wherein the pyrolysis gas has a residence time at a temperature above 370° C. of less than 60 seconds and the weight ratio of catalyst to plastic introduced within the reactor is at least 0.1:30.

2. The process according to claim 1, wherein the pyrolysis gas has a residence time at a temperature above 370° C. of less than 30 seconds.

3. The process according to claim 1, wherein the temperature at which at least a portion of the plastic is converted to waxes is at least 370° C.

4. The process according to claim 1, wherein the reactor is operated at a pressure of below or equal to 1200 mbar.

5. (canceled)

6. The process according to claim 1, wherein the pyrolysis gas is diluted with a diluent, where the diluent is selected from nitrogen, hydrogen, steam, carbon dioxide, combustion gas, hydrocarbon gas and mixtures thereof, and wherein the molar ratio of diluent to pyrolysis products in the pyrolysis gas is above 0.5.

7. (canceled)

8. (canceled)

9. The process according to claim 1, wherein the weight ratio of catalyst to plastic introduced within the reactor is in the range of 0.1:30 to 12:30.

10. The process according to claim 1, wherein the catalyst is or comprises a zeolite-type catalyst.

11. (canceled)

12. The process according to claim 1, wherein the residence time of condensed material in the reactor is between 10 and 600 min.

13. The process according to claim 1, which is conducted continuously.

14. The process according to claim 1, wherein the plastic is waste plastic.

15. Mixture A mixture of hydrocarbons, wherein the hydrocarbons exhibit a cumulative distribution of their number of carbon atoms such that 20≤d20 and 50≥d50;
≥50 mol % of the hydrocarbons are branched hydrocarbons;
and ≤20 mol % of the unsaturated hydrocarbons are alpha-olefins.

16. (canceled)

17. (canceled)

18. The mixture according to claim 15, wherein the hydrocarbons exhibit a cumulative distribution of their number of carbon atoms such that 20≤d20≤40.

19. (canceled)

20. The mixture according to claim 15, wherein the hydrocarbons exhibit a cumulative distribution of their number of carbon atoms such that 50≥d50≥20.

21. The mixture according to claim 15, wherein ≥60 mol % of the hydrocarbons are branched hydrocarbons and ≤15 mol % of the unsaturated hydrocarbons are alpha-olefins.

22. (canceled)

23. (canceled)

24. (canceled)

25. The mixture according to claim 15, having an iodine number in the range of 25 to 100.

26. The mixture according to claim 15, having a drop point of >40° C.

27. (canceled)

28. A wax obtained by a process according to claim 1.

29. The wax according to claim 28, which is a mixture of hydrocarbons, wherein the hydrocarbons exhibit a cumulative distribution of their number of carbon atoms such that 20≤d20 and 50≥d50;
≥50 mol % of the hydrocarbons are branched hydrocarbons;
and ≤20 mol % of the unsaturated hydrocarbons are alpha-olefins.

30. A bituminous mixture, superficial wear coating, asphalt or sealing coating comprising the mixture according to claim 15.

31. A process for producing the mixture according to claim 15 by subjecting plastic to catalytic cracking, the process comprising:
introducing the plastic and a catalyst within a reactor;
allowing at least a portion of the plastic to be converted to waxes, at least part of the waxes being part of the pyrolysis gas formed within the reactor; and
removing a product stream containing said waxes being part of the pyrolysis gas formed within the reactor from the reactor to obtain said mixture;
wherein the pyrolysis gas has a residence time at a temperature above 370° C. of less than 60 seconds and the weight ratio of catalyst to plastic introduced within the reactor is at least 0.1:30.

32. (canceled)
